SEQUENCE LISTING (18 sequences)

SEQ ID NO: 1 - protein, 54 aa, SARS-CoV-2
LFRKSNLKPFERDISTEIYQAGSTPCNGVEGFNCYFPLQSYGFQPTNGVGYQPY

SEQ ID NO: 2 - protein, 29 aa, Artificial sequence
  Synthetic peptide
GYAWNRKRISNCVADYSVLYNSASFSTFK

SEQ ID NO: 3 - protein, 29 aa, Artificial sequence
  Synthetic peptide
  MISC_FEATURE (12)..(12): Cysteine in the peptide chain forms a disulfide bond with cysteine in another non peptide chain
GYAWNRKRISNCVADYSVLYNSASFSTFK

SEQ ID NO: 4 - protein, 38 aa, SARS-CoV-2
ISNCVADYSVLYNSASFSTFKCYGVSPTKLNDLCFTNV

SEQ ID NO: 5 - protein, 38 aa, Artificial sequence; identical to SEQ ID NO: 4
  Synthetic peptide
  MISC_FEATURE (4)..(4): Cysteine in the peptide chain forms a disulfide bond with cysteine in another non peptide chain
  MISC_FEATURE (22)..(22): Cysteine in the peptide chain forms a disulfide bond with cysteine in another non peptide chain
  MISC_FEATURE (34)..(34): Cysteine in the peptide chain forms a disulfide bond with cysteine in another non peptide chain

SEQ ID NO: 6 - protein, 35 aa, SARS-CoV-2
VGGNYNYLYRLFRKSNLKPFERDISTEIYQAGSTP

SEQ ID NO: 7 - protein, 30 aa, SARS-CoV-2
DISTEIYQAGSTPCNGVEGFNCYFPLQSYG

SEQ ID NO: 8 - protein, 20 aa, Artificial sequence
  Synthetic peptide
YFPLQSYGFQPTNGVGYQPY

SEQ ID NO: 9 - protein, 35 aa, SARS-CoV-2
VGGNYNYLYRLFRKSNLKPFERDISTEIYQAGNTP

SEQ ID NO: 10 - protein, 30 aa, SARS-CoV-2
DISTEIYQAGSTPCNGVKGFNCYFPLQSYG

SEQ ID NO: 11 - protein, 20 aa, SARS-CoV-2
YFPLQSYGFQPTYGVGYQPY

SEQ ID NO: 12 - protein, 54 aa, SARS-CoV-2
LFRKSNLKPFERDISTEIYQAGSTPCNGVKGFNCYFPLQSYGFQPTYGVGYQPY

SEQ ID NO: 13 - protein, 22 aa, SARS-CoV-2
QCVNLTTRTQLPPAYTNSFTRG

SEQ ID NO: 14 - protein, 26 aa, SARS-CoV-2
CNDPFLGVYYHKNNKSWMESEFRVYS

SEQ ID NO: 15 - protein, 22 aa, SARS-CoV-2
LHRSYLTPGDSSSGWTAGAAAY

SEQ ID NO: 16 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 17 - protein, 195 aa, SARS-CoV-2 [sequence omitted]

SEQ ID NO: 18 - protein, 69 aa, SARS-CoV-2
SNNLDSKVGGNYNYLYRLFRKSNLKPFERDISTEIYQAGSTPCNGVEGFNCYFPLQSYGFQPTNGVGYQ

(19) United States
(12) Patent Application Publication
(10) Pub. No.: US 2025/0009871 A1
(43) Pub. Date: Jan. 9, 2025

(54) APPLICATION OF NOVEL CORONAVIRUS VACCINE PEPTIDE AND NANOEMULSION PREPARATION THEREOF IN PREVENTION OF NOVEL CORONAVIRUS WILD AND MUTANT STRAINS

(71) Applicants: SHANGHAI INSTITUTE OF MATERIA MEDICA, CHINESE ACADEMY OF SCIENCES, Shanghai (CN); KUNMING INSTITUTE OF ZOOLOGY, CHINESE ACADEMY OF SCIENCES, Yunnan (CN)

(72) Inventors: Likun GONG, Shanghai (CN); Jin REN, Shanghai (CN); Wei HUANG, Shanghai (CN); Yong GAN, Shanghai (CN); Yongtang ZHENG, Yunnan (CN); Jianhua SUN, Shanghai (CN); Xinxin ZHANG, Shanghai (CN); Yiru LONG, Shanghai (CN); Qiuping QIN, Shanghai (CN); Tingting LIU, Shanghai (CN); Feng TANG, Shanghai (CN); Pan YU, Shanghai (CN); Yunqiu MIAO, Shanghai (CN); Yonglong CAI, Guangdong (CN); Mian QIN, Guangdong (CN); Tianzhang SONG, Yunnan (CN)

(21) Appl. No.: 18/564,932

(22) PCT Filed: May 30, 2022
(86) PCT No.: PCT/CN2022/096047

§ 371 Date: Jul. 22, 2024

(30) Foreign Application Priority Data
May 30, 2021 (CN) 202110596132.5

(51) Int. Cl. A61K 39/215; A61K 9/107; A61P 37/04; C07K 14/005

(52) U.S. Cl. CPC A61K 39/215; A61K 9/1075; A61P 37/04; C07K 14/005; C12N 2770/20022; C12N 2770/20034; C12N 2770/20071

(57) ABSTRACT

Disclosed are an application of a coronavirus SARS-CoV-2 vaccine polypeptide, a polypeptide composition and a nanoemulsion preparation thereof in the prevention of coronavirus SARS-CoV-2 wild and mutant strain infections. Specifically, provided is a coronavirus SARS-CoV-2 vaccine polypeptide having an amino acid sequence derived from an S protein of SARS-CoV-2 wild and mutant strains, the vaccine polypeptide can enable the body to generate high-level and durable humoral immune responses against SARS-CoV-2 and to produce high titers of RBD-binding antibodies and neutralizing antibodies that block the binding of RBD to ACE2. The vaccine polypeptide can be used to prevent infections of SARS-CoV-2 wild strain and B.1.1.7, B.1.351, B.1.617, B.1.1.529 and other mutant strains.

### TECHNICAL FIELD

The present invention relates to the field of peptide medicine and peptide vaccines, in particular to the application of a novel coronavirus polypeptide vaccine, a polypeptide composition and nanoemulsion preparation thereof in prevention of novel coronavirus wild and mutant strains.

### BACKGROUND

Novel coronavirus (SARS-CoV-2) spreads on a large scale around the world, and its uncontrolled prevalence seriously threatens global public health security and economic development. The fundamental way to curb the epidemic is to develop and apply safe, economical and efficient novel coronavirus vaccines to achieve herd immunity.

The existing novel coronavirus vaccines mainly adopt the following technical routes: live attenuated vaccines, inactivated vaccines, recombinant viral vector vaccines, recombinant protein vaccines and nucleic acid vaccines. These vaccine technology routes are currently mainly designed for the SARS-CoV-2 wild-type strain.

However, the COVID-19 continues to get out of control, and the virus is also evolving rapidly while spreading rapidly. At present, many virus mutants have emerged and become popular, including D614G mutant, N501.V1 (B.1.1.7) mutant strain, N501.V2 (B.1.351) mutant strain, P.1 mutant strain and Omicron mutant strain (B.1.1.529), etc. Some mutant strains have shown immune escape from existing vaccines and neutralizing antibody drugs. For example, researches have shown that the antibody induced by the inactivated vaccine CoronaVac from Sinovac against the B.1.1.7 mutant strain has significantly decreased, while the neutralizing activity of the novel coronavirus vaccines from Pfizer and AstraZeneca against the B.1.351 mutant strain has decreased 8-9 times. The rapid mutation of novel coronavirus may lead to the difficulty of preventing the infection of novel coronavirus mutants after vaccinating the developed vaccine products, so that it is difficult to achieve herd immunity to control the COVID-19 epidemic.

Therefore, there is an urgent need to develop a safe, efficient and economical second generation of novel coronavirus vaccine, which can respond to the mutations of novel coronavirus, so as to meet the requirements of mass production of vaccines for people around the world at the lowest economic cost, respond to the prevalence of mutant strains, achieve herd immunity worldwide, and effectively control the COVID-19 epidemic.

### SUMMARY OF THE INVENTION

The purpose of the present invention is to provide a novel coronavirus polypeptide vaccine and nanoemulsion vaccine preparation thereof. The vaccine can produce blocking anti-SARS-CoV-2 antibodies in the vaccine recipients, and can deal with the infection of SARS-CoV-2 wild-type and mutant strains.

In the first aspect of the invention, it provides a vaccine polypeptide against novel coronavirus pneumonia, and the vaccine polypeptide has an amino acid sequence derived from the S protein of SARS-CoV-2 wild-type and mutant strains; wherein the vaccine polypeptide is selected from the group consisting of:
(a) a polypeptide having the amino acid sequence shown in any one of SEQ ID NOs: 1-15;
(b) a derivative polypeptide formed by one or more amino acids addition, one or more amino acids substitution, or 1-3 amino acids deletion to the amino acid sequence of the polypeptide in (a), and the derivative polypeptide has essentially identical function to the original polypeptide before derivatization.

In another preferred embodiment, the “essentially identical function” refers that the derivative polypeptide has essentially identical immunogenicity to stimulate cellular and humoral immune responses, as well as to induce primates to produce wild-type RBD specific binding antibodies and neutralizing antibodies that block the binding of wild-type and/or mutant RBD to ACE2.

In another preferred embodiment, the SARS-CoV-2 mutant strains include but are not limited to D614G mutant strain, B.1.1.7 mutant strain, B.1.351 mutant strain, P.1 mutant strain, B.1.617 mutant strain, and B.1.1.529 mutant strain.

In another preferred embodiment, the vaccine polypeptide can induce primates to produce cellular immunity and humoral immunity.

In another preferred embodiment, the vaccine polypeptide can induce primates to produce wild-type RBD specific binding antibodies and neutralizing antibodies that block the binding of wild-type RBD to ACE2.

In another preferred embodiment, the wild-type RBD is the receptor binding domain of the S protein of the SARS-CoV-2 wild-type strain.

In another preferred embodiment, the vaccine polypeptide can stimulate primates to produce mutant RBD specific binding antibodies and neutralizing antibodies that block the binding of mutant RBD to ACE2.

In another preferred embodiment, the mutant RBD is the receptor binding domain of the S protein of the SARS-CoV-2 mutant strain.

In another preferred embodiment, the mutant RBD is a mutant RBD protein formed by the addition of one or more amino acids, substitution of one or more amino acids, or deletion of one or more amino acids on the basis of wild-type RBD.

In another preferred embodiment, the mutations occurring in the mutant RBD include but are not limited to one or more of the following: K417N, K417T, N439K, L452R, Y453F, S447N, E484Q, E484K, and N501Y.

In another preferred embodiment, the antiserum of the vaccine polypeptide immunized primate can block the invasion of SARS-CoV-2 wild-type strain into cells.

In another preferred embodiment, the antiserum of the vaccine polypeptide immunized primate can block the invasion of SARS-CoV-2 mutant strains into cells.

In another preferred embodiment, the primates include humans and non-human primates, such as monkeys.

In another preferred embodiment, the length of the vaccine polypeptide is 8-100 amino acids, preferably 10-80 amino acids.

In another preferred embodiment, the vaccine polypeptide is an artificially synthesized or recombinant vaccine polypeptide.

In another preferred embodiment, the structure of the vaccine polypeptide is as shown in Formula I:

X1-X-X2 (I),
wherein,
(a) X is a core fragment, wherein the sequence of the core fragment is selected from one or more of SEQ ID NOs: 1-15 (see Table A);
(b) X1 and X2 each independently represent none, 1, 2, or 3 amino acids, and the total number of amino acids of X1 and X2 is ≤4, preferably is 3, 2, or 1, and more preferably is 0 or 1;
(c) “-” represents a peptide bond, peptide linker, or other linker (that is, X1 and X, and/or, X and X2 are connected by peptide bonds, peptide linkers (such as a flexible linker consisting of 1-15 amino acids) or other linkers).

In another preferred embodiment, the vaccine polypeptide is selected from Table A:

TABLE A
Vaccine polypeptides

| | | SEQ |
|---|---|---|
| | | ID |
| Peptide ID | Sequence | No: |
| LY54 | LFRKSNLKPFERDISTEIYQAGSTPCNGVEGF | 1 |
| | NCYFPLQSYGFQPTNGVGYQPY | |
| P67 | GYAWNRKRISNCVADYSVLYNSASFSTFK | 2 |
| PSS | GYAWNRKRISNC(C)VADYSVLYNSASFSTFK | 3 |
| LP67 | ISNCVADYSVLYNSASFSTFKCYGVSPTKLND | 4 |
| | LCFTNV | |
| LPSS | ISNC(C)VADYSVLYNSASFSTFKC(C)YGVS | 5 |
| | PTKLNDLC(C)FTNV | |
| MP1 | VGGNYNYLYRLFRKSNLKPFERDISTEIYQAG | 6 |
| | STP | |
| MP2 | DISTEIYQAGSTPCNGVEGENCYFPLQSYG | 7 |
| MP3 | YFPLQSYGFQPTNGVGYQPY | 8 |
| MP477 | VGGNYNYLYRLFRKSNLKPFERDISTEIYQAG | 9 |
| | NTP | |
| MP484 | DISTEIYQAGSTPCNGVKGFNCYFPLQSYG | 10 |
| MP501 | YFPLQSYGFQPTYGVGYQPY | 11 |
| LY54mut | LFRKSNLKPFERDISTEIYQAGSTPCNGVKGF | 12 |
| | NCYFPLQSYGFQPTYGVGYQPY | |
| NTD1 | QCVNLTTRTQLPPAYTNSFTRG | 13 |
| NTD2 | CNDPFLGVYYHKNNKSWMESEFRVYS | 14 |
| NTD3 | LHRSYLTPGDSSSGWTAGAAAY | 15 |

wherein, the “C(C)” in SEQ ID Nos: 3 and 5 indicates that the cysteine on the peptide chain forms a disulfide bond with another cysteine on the non-peptide chain.

Specifically, the thiol group of cysteine on the main chain of PSS forms a disulfide bond structure with one thiol group of cysteine, and the structure and schematic diagram are shown in FIG. 1.

The three cysteine thiol groups on the main chain of LPSS (also known as LP67-1) form disulfide bond structures with the thiol groups of cysteine, respectively. The structure and schematic diagram are shown in FIG. 2.

In another preferred embodiment, the three vaccine polypeptides shown in SEQ ID Nos: 6, 7, and 8 are used in combination, among which any one, two, or three of SEQ ID Nos: 6-8 can also be introduced with corresponding mutation sites (such as the mutation sites in SEQ ID Nos: 6-8 shown in FIG. 19) in a timely manner based on the variation of SARS-CoV-2, to form a variant strain (or mutant strain) that can respond to SARS-CoV-2 in a timely manner, thereby improving the protective effect of the vaccine of the present invention against mutant strains.

A representative set (or combination) of vaccine polypeptides that are more suitable for dealing with mutant strains includes: vaccine polypeptides shown in SEQ ID Nos: 9, 10, and 11 (each one is introduced with mutant amino acids of mutant strains).

Furthermore, one or more vaccine polypeptides of the present invention which are against wild-type virus strains and are as shown in SEQ ID Nos: 6, 7, and 8, may also be combined with one or more vaccine polypeptides against mutant strains (the vaccine polypeptides as shown in SEQ ID Nos: 9, 10, and 11), to form a vaccine polypeptide set such as consisting of the vaccine polypeptides shown in SEQ ID Nos: 9, 7, and 11; or a vaccine peptide set consisting of the vaccine polypeptides shown in SEQ ID Nos: 9, 10, and 8.

In another preferred embodiment, X1 or X2 each independently represents none, K, C, G, L, or A.

In another preferred embodiment, X1 is none, K, or C.

In another preferred embodiment, X2 is none, K, or C.

In another preferred embodiment, the vaccine polypeptide has at least one T cell epitope and/or at least one B cell epitope of the SARS-CoV-2 S protein.

In another preferred embodiment, the vaccine polypeptide has at least one T cell epitope and/or at least one B cell epitope of the RBD region of the SARS-CoV-2 S protein.

In another preferred embodiment, the vaccine polypeptide has at least one T cell epitope and/or at least one B cell epitope of the RBM region of the SARS-CoV-2 S protein.

In another preferred embodiment, the vaccine polypeptide has at least one T cell epitope and/or at least one B cell epitope of the NTD region of the SARS-CoV-2 S protein.

In another preferred embodiment, the vaccine polypeptide has at least one T cell epitope, preferably 1, 2, 3 or 4 T cell epitopes, and more preferably 1 or 2 T cell epitopes.

In another preferred embodiment, the vaccine polypeptide has at least one B cell epitope, preferably 1, 2, 3 or 4 B cell epitopes, and more preferably 1 or 2 B cell epitopes.

In another preferred embodiment, the vaccine polypeptide has 1-2 T cell epitopes and 0-2 B cell epitopes, preferably 1-2 T cell epitopes and 0-1 B cell epitope.

In the second aspect of the present invention, it provides an isolated peptide set, comprising at least two vaccine polypeptides against the novel coronavirus pneumonia according to the first aspect of the present invention.

In another preferred embodiment, the peptide set contains at least 2-15 kinds (such as 2, 3, 4, 5, 6, 7, 8, 9, 10, 11, 12, 13, 14 or 15 kinds) of the vaccine polypeptides.

In another preferred embodiment, the peptide set comprises vaccine polypeptides selected from the group consisting of:
(Z1) 1 or 2 vaccine polypeptides selected from SEQ ID Nos: 1 and 12, and 1, 2, 3, or 4 vaccine polypeptides selected from SEQ ID Nos: 2-5;
(Z2) 1 or 2 vaccine polypeptides selected from SEQ ID Nos: 6 and 9, 1 or 2 vaccine polypeptides selected from SEQ ID Nos: 7 and 10, and 1 or 2 vaccine polypeptides selected from SEQ ID Nos: 8 and 11;
(Z3) one or more (such as 1, 2, 3, 4 or 5) vaccine polypeptides selected from SEQ ID Nos: 1-12, and 1, 2 or 3 vaccine polypeptides selected from SEQ ID Nos: 13-15.

In another preferred embodiment, the peptide set comprises vaccine polypeptides selected from the group consisting of (or the peptide set is composed of vaccine polypeptides selected from the group consisting of):
(s1) the vaccine polypeptide shown in SEQ ID No: 1, and the vaccine polypeptide shown in SEQ ID No: 2;
(s2) 1 or 2 vaccine polypeptides selected from SEQ ID Nos: 1 and 12, and 1, 2, 3 or 4 vaccine polypeptides selected from SEQ ID Nos: 2-5, and the peptide set at least comprises one or more vaccine polypeptides selected from SEQ ID Nos: 3, 4, 5, and 12;
(s3) the vaccine polypeptides shown in SEQ ID Nos: 6, 7 and 8;
(s4) the vaccine polypeptides shown in SEQ ID Nos: 9, 10 and 11;
(s5) the vaccine polypeptides shown in SEQ ID Nos: 6, 10 and 11;
(s6) the vaccine polypeptides shown in SEQ ID Nos: 9, 7 and 11;
(s7) the vaccine polypeptides shown in SEQ ID Nos: 9, 10 and 8;
(s8) the vaccine polypeptides shown in SEQ ID Nos: 2, 9, 10 and 11;
(s9) the vaccine polypeptides shown in SEQ ID Nos: 2, 6, 7 and 8.

In the third aspect of the present invention, it provides a pharmaceutical composition comprising the vaccine polypeptide against novel coronavirus pneumonia according to the first aspect of the present invention or the peptide set according to the second aspect of the present invention and a pharmaceutically acceptable carrier.

In another preferred embodiment, the pharmaceutical composition is a vaccine composition.

In another preferred embodiment, the vaccine composition is monovalent or multivalent.

In another preferred embodiment, the pharmaceutical composition further comprises an adjuvant, which includes but is not limited to an aluminum salt, Titermax, an emulsion, a liposome, and a viral vector.

In another preferred embodiment, the pharmaceutical composition includes a single prescription preparation, a compound prescription preparation, or a synergistic drug.

In another preferred embodiment, the dosage form of the pharmaceutical composition is liquid, solid, or gel.

In another preferred embodiment, the pharmaceutical composition is administered in a manner selected from the group consisting of: subcutaneous injection, intradermal injection, intramuscular injection, intravenous injection, intraperitoneal injection, microneedle injection, oral administration, or oral and nasal spray and atomization inhalation.

In the fourth aspect of the present invention, it provides a vaccine formulation, which comprises the vaccine polypeptide against novel coronavirus pneumonia according to the first aspect of the present invention, or the peptide set according to the second aspect of the present invention, or the pharmaceutical composition according to the third aspect of the present invention, and the vaccine formulation is preferably a nanoemulsion formulation.

In another preferred embodiment, the vaccine formulation comprises a nanoemulsion based on squalene and an emulsifier.

In another preferred embodiment, the squalene is derived from shark liver, and the emulsifier is phospholipid, or polysorbate 80, or a combination of polysorbate 80 and phospholipids, as well as one or more of sucrose esters, citric acid fatty acid glycerides, fatty acid glycerides, fatty acid sorbitans, cyclodextrins, polyoxyethylene fatty acid esters, etc.

In another preferred embodiment, the vaccine formulation comprises 0-20% of acceptable raw materials in other injection forms. The vaccine adjuvant composition comprises α-tocopherol, and the content of the α-tocopherol does not exceed 15 wt %; the vaccine adjuvant composition comprises a block copolymer, which may be one or more of methoxy polyethylene glycol polylactic acid hydroxyacetic acid copolymer, methoxy polyethylene glycol polylactic acid-hydroxyacetic acid and poloxamer, with a content not exceeding 5%; and it further contains an isotonic regulator, which is of 0.1-8% (w/w); the vaccine adjuvant combination further contains a pH regulator, which does not exceed 1% (w/w).

In the fifth aspect of the present invention, it provides a use of the vaccine polypeptide against novel coronavirus pneumonia according to the first aspect of the present invention, or the peptide set according to the second aspect of the present invention, or the pharmaceutical composition according to the third aspect, or the formulation according to the fourth aspect of the present invention, in the manufacture of a medicament for the prevention of coronavirus SARS-CoV-2 wild-type and mutant strains infection or related diseases thereof.

In another preferred embodiment, the coronavirus SARS-CoV-2 related disease is selected from the group consisting of: respiratory infection, pneumonia and its complications, and a combination thereof.

In another preferred embodiment, the coronavirus SARS-CoV-2 related disease is novel coronavirus pneumonia (COVID-19).

In the sixth aspect of the present invention, it provides a cell preparation, comprising (a) immune cells which are immune activated by the vaccine polypeptide according to the first aspect of the present invention or the peptide set according to the second aspect of the present invention; and (b) a pharmaceutically acceptable carrier.

In another preferred embodiment, the immune cells are selected from the group consisting of: dendritic cells, natural killer (NK) cells, lymphocytes, monocytes/macrophages, granulocytes, and a combination thereof.

In another preferred embodiment, the activation is in vitro activation.

In another preferred embodiment, the in vitro activation includes: culturing the immune cells for a period of time (such as 6-48 hours) in the presence of the vaccine polypeptide to obtain the immune activated immune cells.

In another preferred embodiment, the cell preparation is a liquid preparation comprising living cells.

In another preferred embodiment, the cell preparation is reinfused through intravenous administration.

In the seventh aspect of the present invention, it provides a method for generating an immune response to coronavirus SARS-CoV-2 wild-type and mutant strains, which comprises the step of: administering the vaccine polypeptide according to the first aspect of the present invention, the peptide set according to the second aspect of the present invention, the pharmaceutical composition according to the third aspect of the present invention, or the formulation according to the fourth aspect of the present invention, to a subject in need thereof.

In another preferred embodiment, the subject comprises human or non-human mammals.

In another preferred embodiment, the non-human mammals comprise non-human primates (such as monkeys).

In another preferred embodiment, the method induces the production of neutralizing antibodies against the coronavirus SARS-CoV-2 wild-type and mutant strains in the subject.

In another preferred embodiment, the neutralizing antibodies block the binding of coronavirus SARS-CoV-2 wild-type and mutant strains to human ACE2 protein.

In the eighth aspect of the present invention, it provides a fusion protein, comprising a carrier protein and the vaccine polypeptide according to the first aspect of the present invention fused to the fusion protein.

In another preferred embodiment, the fusion protein has a structure of Formula IIIa or IIIb:

P1-P2 (IIIa)

P2-P1 (IIIb)

wherein, P1 is the vaccine polypeptide according to the first aspect of the present invention, and P2 is the carrier protein.

In another preferred embodiment, P1 may be a single vaccine polypeptide, or a tandem vaccine polypeptide formed by multiple identical or different vaccine polypeptides (or antigen polypeptides).

In the ninth aspect of the present invention, it provides a pharmaceutical composition, which comprises (a) the fusion protein according to the seventh aspect of the present invention or immune cells which are immune activated by the fusion protein; and (b) a pharmaceutically acceptable carrier.

In the tenth aspect of the present invention, it provides a use of the fusion protein according to the seventh aspect of the present invention or the pharmaceutical composition according to the eighth aspect of the present invention, in the manufacture of a medicament for the prevention of coronavirus SARS-CoV-2 infection or related diseases thereof.

It should be understood that within the scope of the present invention, each technical features of the present invention described above and in the following (such as examples) may be combined with each other to form a new or preferred technical solution, which is not listed here due to space limitations.

### DESCRIPTION OF THE DRAWINGS

FIG. 1 shows the specific structure of polypeptide PSS.

FIG. 2 shows the specific structure of polypeptide LPSS.

FIG. 3 shows the MHC class II molecular epitope analysis results of the RBD region of SARS-CoV-2.

FIG. 4 shows the MHC class I molecular epitope analysis results of the RBD region of SARS-CoV-2.

FIG. 5 shows the localization of vaccine polypeptides LY54, LY54mut, P67, PSS, LPSS, and LP67 derived from wild-type RBD in the RBD protein.

FIG. 6 shows the localization of vaccine polypeptides MP1, MP2, MP3, MP477, MP484, and MP501 derived from mutant RBD in the RBD protein.

FIG. 7 shows the production of high-level RBD specific binding antibodies in cynomolgus monkeys immunized with vaccine polypeptides after 34 days.

FIG. 8 shows the production of high-level neutralizing antibody that can block the interaction of RBD with ACE2 in cynomolgus monkeys immunized with vaccine polypeptides after 34 days.

FIG. 9 shows that the RBD specific binding antibodies are still in high level in cynomolgus monkeys immunized with vaccine polypeptides after 105 days.

FIG. 10 shows that the neutralizing antibodies that can block the interaction of RBD with ACE2 are still in high level in cynomolgus monkeys immunized with vaccine polypeptides after 105 days.

FIG. 11 shows the production of high-level binding antibodies in cynomolgus monkeys immunized with polypeptide nanoemulsion formulation after 42 days.

FIG. 12 shows the production of high-level neutralizing antibodies that can block the interaction of RBD with ACE2 in cynomolgus monkeys immunized with polypeptide nanoemulsion formulation after 42 days.

FIG. 13 shows that the antiserum of cynomolgus monkeys immunized with the polypeptide nanoemulsion formulation has high neutralizing activity in blocking the invasion of the wild-type strain pseudovirus into cells.

FIG. 14 shows that the antiserum of cynomolgus monkeys immunized with the polypeptide nanoemulsion formulation has high neutralizing titers of blocking the invasion of the wild-type strain pseudovirus into cells.

FIG. 15 shows the production of high-level mutant RBD specific binding antibodies in cynomolgus monkeys immunized with polypeptide nanoemulsion formulation.

FIG. 16 shows that the antiserum of cynomolgus monkeys immunized with the polypeptide nanoemulsion formulation has high neutralizing activity in blocking the invasion of the mutant strain B.1.1.7 pseudovirus into cells.

FIG. 17 shows the production of high-level specific binding antibodies against key RBD mutation sites in B.1.351 and P.1 mutant strains in cynomolgus monkeys immunized with a combination of vaccine polypeptides MP477, MP484, MP501, and P67 derived from mutant RBD.

FIG. 18 shows the production of high-level specific binding antibodies against key RBD mutation sites in B.1.617 mutant strain in cynomolgus monkeys immunized with a combination of vaccine polypeptides MP477, MP484, MP501, and P67 derived from mutant RBD.

FIG. 19 shows the mutant amino acid sites and mutant types of S protein regions of several novel coronavirus mutant strains compared with those of the wild-type strain.

FIG. 20 shows that the antiserum of cynomolgus monkeys immunized with the polypeptide nanoemulsion formulation has high neutralizing activity in blocking the invasion of the mutant strain B.1.1.529 pseudovirus into cells.

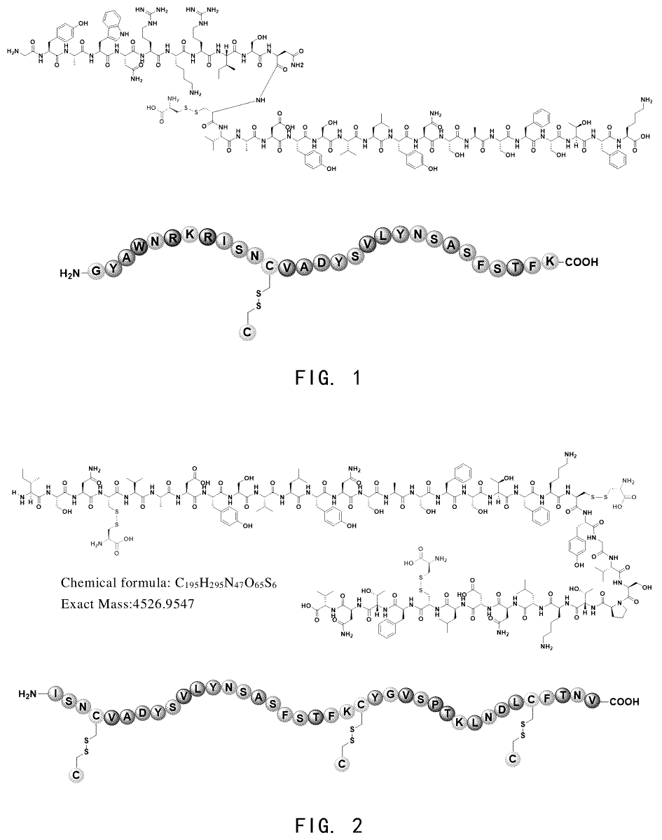

### DETAILED DESCRIPTION

Through extensive and in-depth research, the inventors analyzed the immunogenicity of S protein of wild-type and mutant strains based on sequence analysis of S proteins of SARS-CoV-2 wild-type strain and various mutant strains and structural analysis of the interaction between S proteins and host ACE2, and identified the key regions of interaction between S proteins of wild-type and mutant strains and host ACE2 protein. The present invention screened and determined vaccine polypeptides that could effectively induce an immune response against the coronavirus SARS-CoV-2 in primate organisms. Experiments have shown that the vaccine polypeptide of the present invention can induce a high-level and persistent humoral immune response against SARS-CoV-2 in cynomolgus monkeys. The antiserum of cynomolgus monkeys immunized with the vaccine polypeptide and polypeptide nanoemulsion vaccine formulation of the present invention has high titers of RBD binding antibodies and neutralizing antibodies that block the binding of RBD to ACE2, and has shown a high level of blocking effect on both SARS-CoV-2 pseudovirus model and live virus model. At the same time, compared with the wild-type RBD, the binding activity of the cynomolgus monkey antiserum against various RBD mutant proteins such as S477N, E484K and N501Y is not affected, and high-level blocking effect is shown in the B.1.1.7 mutant pseudovirus model, indicating that the polypeptide vaccine of the present invention can be used to prevent the infection of novel coronavirus wild-type strain and various mutant strains such as B.1.1.7, B.1.351 and B.1.1.529. The present invention has been completed on this basis.

### Term

### Coronavirus SARS-CoV-2

Coronavirus (CoV) belongs to the Nidovirales Coronaviridae, which is an enveloped positive-strand RNA virus, and its subfamily includes four genera, α, β, δ, and γ. Among the currently known coronaviruses that infect humans, HCoV-229E and HCoV-NL63 belong to the α genus coronavirus, and HCoV-OC43, SARS-CoV, HCoV-HKU1, MERS-CoV and SARS-CoV-2 are all β genus coronavirus.

The novel coronavirus (SARS-CoV-2) that broke out at the end of 2019 has about 80% similarity with SARS-CoV and 40% similarity with MERS-CoV. It also belongs to the β genus coronavirus.

The genome of this type of virus is a single-stranded positive-stranded RNA, and the virus is one of those RNA viruses with the largest genome, which encodes replicase, spike protein, membrane protein, envelope protein, and nucleocapsid protein, etc. In the initial stage of virus replication, the genome is translated into two peptide chains with length of several thousand amino acids, that is the precursor polyprotein, and then the precursor protein is cleaved by proteases to produce non-structural proteins (such as RNA polymerase and helicase) and structural proteins (such as spike proteins) and accessory proteins.

The S protein is a major structural protein of the coronavirus SARS-CoV-2, wherein, RBD is responsible for binding to human ACE2 receptor, and the RBM region comprises a motif that binds to human ACE2. The amino acid sequence of a typical S protein is shown in SEQ ID No: 16.

>S protein
(SEQ ID No: 16)
MFVFLVLLPLVSSQCVNLTTRTQLPPAYTNSFTRGVYYPDKVFRSSVL
HSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVY
YHKNNKSWMESEFRVYSSANNCTFEYVSQPFLMDLEGKQGNFKNLREF
VFKNIDGYFKIYSKHTPINLVRDLPQGFSALEPLVDLPIGINITRFQT
LLALHRSYLTPGDSSSGWTAGAAAYYVGYLQPRTFLLKYNENGTITDA
VDCALDPLSETKCTLKSFTVEKGIYQTSNFRVQPTESIVRFPNITNLC
PFGEVFNATRFASVYAWNRKRISNCVADYSVLYNSASFSTFKCYGVSP
TKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPK
KSTNLVKNKCVNFNFNGLTGTGVLTESNKKFLPFQQFGRDIADTTDAV
RDPQTLEILDITPCSFGGVSVITPGTNTSNQVAVLYQDVNCTEVPVAI
HADQLTPTWRVYSTGSNVFQTRAGCLIGAEHVNNSYECDIPIGAGICA
SYQTQTNSPRRARSVASQSIIAYTMSLGAENSVAYSNNSIAIPTNFTI
SVTTEILPVSMTKTSVDCTMYICGDSTECSNLLLQYGSFCTQLNRALT
GIAVEQDKNTQEVFAQVKQIYKTPPIKDFGGFNFSQILPDPSKPSKRS
FIEDLLFNKVTLADAGFIKQYGDCLGDIAARDLICAQKFNGLTVLPPL
LTDEMIAQYTSALLAGTITSGWTFGAGAALQIPFAMQMAYRFNGIGVT
QNVLYENQKLIANQFNSAIGKIQDSLSSTASALGKLQDVVNQNAQALN
TLVKQLSSNFGAISSVLNDILSRLDKVEAEVQIDRLITGRLQSLQTYV
TQQLIRAAEIRASANLAATKMSECVLGQSKRVDFCGKGYHLMSFPQSA
PHGVVFLHVTYVPAQEKNFTTAPAICHDGKAHFPREGVFVSNGTHWFV
TQRNFYEPQIITTDNTFVSGNCDVVIGIVNNTVYDPLQPELDSFKEEL
DKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCC
SCGSCCKFDEDDSEPVLKGVKLHYT

The RBD region of the coronavirus SARS-CoV-2 is located at positions 333-527 of the S protein, and a representative amino acid sequence is shown in positions 333-527 of SE ID No: 16.

>RBD (333-527)
(SEQ ID NO: 17)
TNLCPFGEVFNATRFASVYAWNRKRISNCVADYSVLYNSASFSTFKCYGV
SPTKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPCNG
VEGFNCYFPLQSYGFQPTNGVGYQPYRVVVLSFELLHAPATVCGP

The RBM region of the coronavirus SARS-CoV-2 is located at positions 438-506 of the S protein, and a representative amino acid sequence is shown in positions 438-506 of SEQ ID No: 17.

>RBM (438-506)
(SEQ ID NO: 18)
SNNLDSKVGGNYNYLYRLFRKSNLKPFERDISTEIYQAGSTPCNGVEGE
NCYFPLQSYGFQPTNGVGYQ

It should be understood that in the present invention, the S protein, RBD region and RBM region all include wild-type and mutant type.

At present, various mutant strains of SARS-CoV-2 different from the wild-type strain Wuhan-Hu-1 have emerged and are widely prevalent. For example, a mutant strain called B.1.1.7 or 501Y.V1 has been detected in many countries around the world and may have stronger infectivity and increased risk of death. Another mutant strain is called B.1.351 or 501Y.V2, which has begun to spread outside of South Africa and exhibits strong immune escape capabilities from vaccines and neutralizing antibodies. The mutant strain P.1 seems to have a stronger immune escape ability than mutant strain B.1.351. The Omicron (B.1.1.529) mutant strain shows more significant immune escape and transmission abilities.

The comparison of mutation locations and types in the S protein region between some mutant strains and wild strain is shown in FIG. 19.

### Vaccine Polypeptide

In the present invention, “epitope peptide of the present invention”, “vaccine polypeptide of the present invention”, “antigen polypeptide of the present invention”, “polypeptide of the present invention” can be used interchangeably, and refer to the vaccine polypeptide according to the first aspect of the present invention. It should be understood that the term includes not only one kind of vaccine polypeptide of the present invention, but also peptide sets (or peptide combinations) formed by multiple vaccine polypeptides of the present invention.

In the present invention, vaccine polypeptides also include other forms, such as pharmaceutically acceptable salts, conjugates, or fusion proteins.

In the present invention, the vaccine polypeptide includes a derivative polypeptide formed by one or more (e.g., 1-5, preferably 1-3) amino acids addition, one or more (e.g., 1-5, preferably 1-3) amino acids substitution, and/or 1-3 amino acids deletion to any one of sequences shown in SEQ ID Nos: 1-15, and the derivative polypeptide has essentially the same function as the original polypeptide before derivatization.

Preferably, the vaccine polypeptide includes 1-3 amino acids addition (preferably the N-terminal or C-terminal addition), and/or 1-2 amino acids substitution (preferably conservative amino acid substitution) to any one of sequences shown in SEQ ID Nos: 1-15, and still has essentially the same function as the original polypeptide before derivatization.

Preferably, the conservative amino acid substitution is performed according to Table C.

TABLE C

| Initial | Representative | Preferred |
|---|---|---|
| residue | substitution | substitution |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

In another preferred embodiment, the three vaccine polypeptides shown in SEQ ID Nos: 6, 7, and 8 are used in combination, among which any one, two, or three of SEQ ID Nos: 6-8 can also be introduced with corresponding mutation sites (such as the mutation sites in SEQ ID Nos: 6-8 shown in FIG. 19) in a timely manner based on the variation of SARS-CoV-2, to form a variant strain (or mutant strain) that can respond to SARS-CoV-2 in a timely manner, thereby improving the protective effect of the vaccine of the present invention against mutant strains.

A representative set (or combination) of vaccine polypeptides that are more suitable for dealing with mutant strains includes vaccine polypeptides shown in SEQ ID Nos: 9, 10, and 11 (each one is introduced with mutant amino acids of mutant strains).

Furthermore, one or more vaccine polypeptides of the present invention which are against wild-type virus strains and are as shown in SEQ ID Nos: 6, 7, and 8, may also be combined with one or more vaccine polypeptides against mutant strains (the vaccine polypeptides as shown in SEQ ID Nos: 9, 10, and 11), to form a vaccine polypeptide set such as consisting of the vaccine polypeptides shown in SEQ ID Nos: 9, 7, and 11; or a vaccine peptide set consisting of the vaccine polypeptides shown in SEQ ID Nos: 9, 10, and 8.

As used herein, the peptide set described by the term “peptide set” consists of at least two kinds of vaccine polypeptides of the present invention or derivative polypeptides thereof.

Preferably, the peptide set of the present invention comprises at least 2, 3, 4, 5, 6, 7, 8, 9, 10, 11, 12, 13, 14 or 15 kinds of vaccine polypeptides or their derivative polypeptides (including coupled peptides) selected from the first aspect of the present invention; more preferably, the peptide set comprises at least 2, 3, 4, 5, 6, 7, 8, 9, 10, 11, 12, 13, 14 or 15 kinds of vaccine polypeptides selected from SEQ ID Nos: 1-15 or their derivative polypeptides. In addition, the peptide set may also include antigen peptides or proteins of other coronavirus SARS-CoV-2 besides SEQ ID Nos: 1-15.

In another preferred embodiment, the peptide set comprises vaccine polypeptides selected from the group consisting of:
(Z1) 1 or 2 vaccine polypeptides selected from SEQ ID Nos: 1 and 12, and 1, 2, 3, or 4 vaccine polypeptides selected from SEQ ID Nos: 2-5;
(Z2) 1 or 2 vaccine polypeptides selected from SEQ ID Nos: 6 and 9, 1 or 2 vaccine polypeptides selected from SEQ ID Nos: 7 and 10, and 1 or 2 vaccine polypeptides selected from SEQ ID Nos: 8 and 11;
(Z3) one or more (such as 1, 2, 3, 4 or 5) vaccine polypeptides selected from SEQ ID Nos: 1-12, and 1, 2 or 3 vaccine polypeptides selected from SEQ ID Nos: 13-15.

In another preferred embodiment, the peptide set comprises vaccine polypeptides selected from the group consisting of (or the peptide set is composed of vaccine polypeptides selected from the group consisting of):
(s1) the vaccine polypeptide shown in SEQ ID No: 1, and the vaccine polypeptide shown in SEQ ID No: 2;
(s2) 1 or 2 vaccine polypeptides selected from SEQ ID Nos: 1 and 12, and 1, 2, 3 or 4 vaccine polypeptides selected from SEQ ID Nos: 2-5, and the peptide set at least comprises one or more vaccine polypeptides selected from SEQ ID Nos: 3, 4, 5, and 12;
(s3) the vaccine polypeptides shown in SEQ ID Nos: 6, 7 and 8;
(s4) the vaccine polypeptides shown in SEQ ID Nos: 9, 10 and 11;
(s5) the vaccine polypeptides shown in SEQ ID Nos: 6, 10 and 11;
(s6) the vaccine polypeptides shown in SEQ ID Nos: 9, 7 and 11;
(s7) the vaccine polypeptides shown in SEQ ID Nos: 9, 10 and 8;
(s8) the vaccine polypeptides shown in SEQ ID Nos: 2, 9, 10 and 11;
(s9) the vaccine polypeptides shown in SEQ ID Nos: 2, 6, 7 and 8.

As used herein, “isolated” refers to the separation of a substance from its original environment (if it is a natural substance, the original environment is the natural environment). For example, the polypeptide of living cells in the natural state is not separated or purified, but the same polypeptide is separated and purified if it is separated from other substances that exist in the natural state.

As used herein, “isolated peptide” means that the polypeptide of the present invention substantially does not contain other naturally related proteins, lipids, carbohydrates or other substances. Those skilled in the art can use standard protein purification techniques to purify the polypeptide of the present invention. Basically, the purified polypeptide (fusion protein) can produce a single main band on a non-reducing polyacrylamide gel.

The polypeptide of the present invention may be a recombinant polypeptide or a synthetic polypeptide, preferably a synthetic polypeptide.

In the present invention, when the sequence of the vaccine polypeptide is short (such as ≤70aa, more preferably ≤60aa), the relevant peptide sequence may be directly synthesized by chemical methods.

When the sequence of the vaccine polypeptide is longer or the vaccine polypeptide is provided in the form of a fusion protein, the recombinant method may also be used to obtain the related peptide sequence in large quantities. This usually involves cloning the coding sequence of the antigen polypeptide or its fusion protein into a vector, and then transferring it into cells, followed by isolating the relevant antigen polypeptide or fusion protein from the proliferated host cells via conventional methods.

### Pharmaceutical Composition and Mode of Administration

The present invention also provides a pharmaceutical composition. The pharmaceutical composition of the present invention may be therapeutic or prophylactic (e.g., a vaccine). The pharmaceutical composition of the present invention includes an effective amount of the vaccine polypeptide or peptide set of the present invention, or immune cells activated with the vaccine polypeptide (e.g., dendritic cells sensitized with the vaccine polypeptide of the present invention or T cells induced by the dendritic cells), and at least one pharmaceutically acceptable carrier, diluent or excipient.

In the present invention, these (vaccine) compositions comprise immune antigens (including the vaccine polypeptides, peptide sets or derivatives thereof of the present invention), and are usually combined with “pharmaceutically acceptable carriers”, including any carrier that itself does not induce the production of antibodies that are harmful to the individual receiving the composition. Examples of suitable carriers include (but are not limited to) proteins, lipid aggregates (such as oil droplets or liposomes) and so on. These carriers are well known to those of ordinary skill in the art. In addition, these carriers may function as immunostimulants (“adjuvants”).

In addition, the (vaccine) composition of the present invention may also comprise additional adjuvants. Representative vaccine adjuvants include (but are not limited to) the following types: inorganic adjuvants, such as aluminum hydroxide, alum, etc.; synthetic adjuvants, such as synthetic double-stranded polynucleotides (double-stranded polyadenylic acid, uridylic acid), levamisole, isoprinosine, etc.; oil agents, such as Freund's adjuvant, peanut oil emulsification adjuvant, mineral oil, vegetable oil, etc.

Generally, the vaccine composition or immunogenic composition may be made into an injectable agent, for example, a liquid solution or suspension; it may also be made into a solid form suitable for being formulated into a solution or suspension or a liquid excipient before injection. The preparation may also be emulsified or encapsulated in liposomes to enhance the adjuvant effect.

The composition may be made into a unit or multiple dosage form. Each dosage form comprises a predetermined amount of active substances calculated to produce the desired therapeutic effect, and suitable pharmaceutical excipients.

The formulated pharmaceutical composition may be administered by conventional routes, including (but not limited to): intravenous, intramuscular, intraperitoneal, subcutaneous, intradermal, oral, or topical administration.

When using the (vaccine) composition, a safe and effective amount of the vaccine polypeptide or peptide set of the present invention is administered to a human, wherein the safe and effective amount is usually at least about 1 μg peptide/kg body weight, and in most cases not more than about 8 mg peptide/kg body weight, preferably the dose is about 1 μg-1 mg peptide/kg body weight. Of course, the specific dosage should also consider factors such as the administration route and the patient's health status, which are all within the skill range of a skilled physician.

The main advantages of the present invention include:
(a) The vaccine polypeptide and polypeptide nanoemulsion vaccine formulation used in the present invention can produce high-level and persistent wild-type and multiple mutant RBD specific binding antibodies in the body of primates, as well as neutralizing antibodies that block the binding of wild-type and mutant RBD to ACE2.
(b) The antiserum of primates immunized with the polypeptide nanoemulsion vaccine formulation used in the present invention has a high level of neutralizing activity in blocking the invasion of wild-type pseudoviruses and live viruses into cells.
(c) The antiserum of primates immunized with the polypeptide nanoemulsion vaccine formulation used in the present invention has a high level of neutralizing activity in blocking the invasion of the mutant strain B.1.1.7 pseudoviruses into cells.

The present invention is further explained below in conjunction with specific examples. It should be understood that these examples are only for illustrating the present invention and not intend to limit the scope of the present invention. The conditions of the experimental methods not specifically indicated in the following examples are usually in accordance with conventional conditions as described in Sambrook et al., Molecular Cloning: A Laboratory Manual (New York: Cold Spring Harbor Laboratory Press, 1989), or according to the conditions recommended by the manufacturer. Unless otherwise stated, percentages and parts are calculated by weight.

### Example 1 Determination of Antigen Polypeptides Based on the S Protein of SARS-CoV-2 Wild-Type and Mutant Strains

In this example, the inventors used the RBD region in the S protein of SARS-CoV-2 wild-type and mutant strains that interacts with human ACE2 as the analysis object to predict its immunodominant antigen sites.

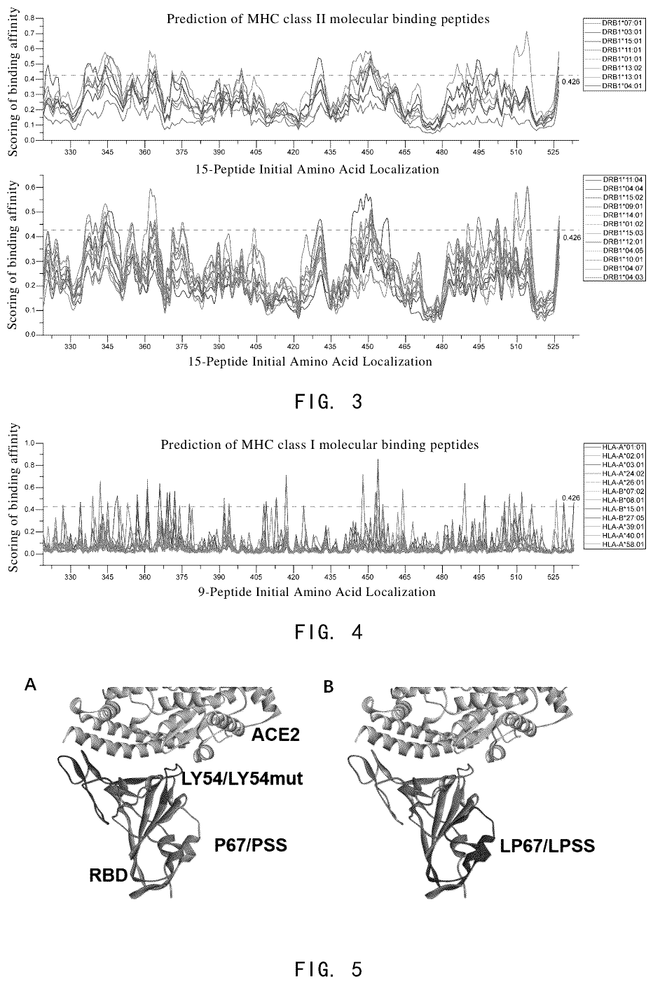

The inventors used software to predict and comprehensively analyze the immunogenicity of RBD sequences, mainly including MHC class II molecular binding epitopes (FIG. 3) and MHC class I molecular binding epitopes (FIG. 4).

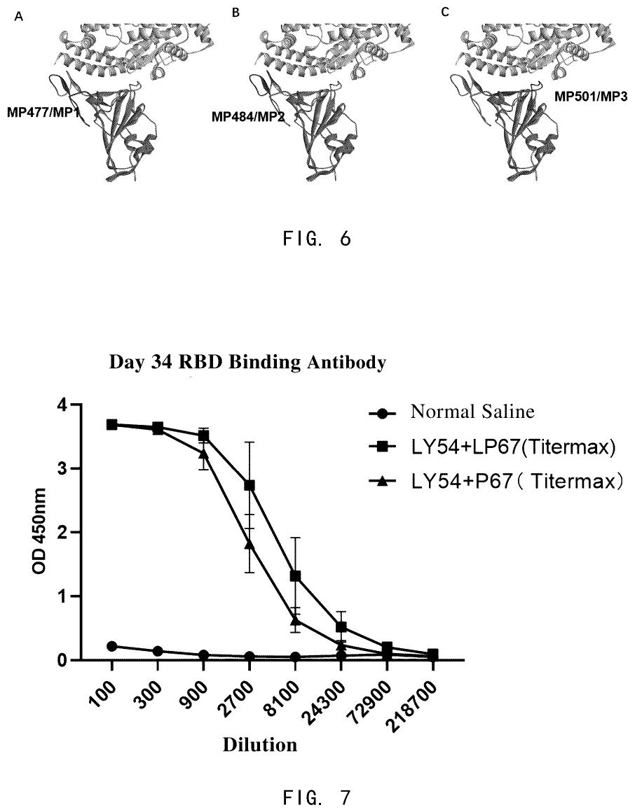

Based on the immunogenicity analysis of the S protein of SARS-CoV-2 wild-type and mutant strains, as well as the spatial location of peptide segments, etc., the inventors ultimately screened 15 polypeptides with amino acid sequences shown in SEQ ID NOs: 1-15. The screened vaccine polypeptides mainly correspond to the RBD region and adjacent regions. Representative localization on the RBD of the SARS-CoV-2 wild-type and mutant strains is shown in FIGS. 5 and 6, respectively.

### Example 2 Preparation of Polypeptides

In this example, the solid-phase polypeptide synthesizer was used to prepare the polypeptides shown in SEQ ID NOs: 1-15. The synthesized polypeptides were identified and purified by HPLC to ensure their correct properties and purity of over 95%.

### Example 3 Immune Experiments on Cynomolgus Monkeys with Polypeptide Vaccines

In this example, the immune effects of polypeptide vaccines against SARS-CoV-2 were further verified in cynomolgus monkeys.

Immune formulations were prepared using TiterMax adjuvant with (a) LY54+P67, (b) LY54+LP67, and (c) LY54, respectively. The immunization procedure involved administering polypeptide vaccine formulations to cynomolgus monkeys by intramuscular injection on day 0 and day 14. The immune dose of vaccine polypeptides was 1 mg per animal. The serum of immunized cynomolgus monkeys was used for the detection of RBD binding antibodies and neutralizing antibodies, as well as for the invasion blocking test of SARS-CoV-2 pseudovirus and live virus.

### Example 4 Detection of Polypeptide Vaccine-Induced Humoral Immune Response

To evaluate the level of humoral immune response induced by polypeptide vaccines in cynomolgus monkeys, it is necessary to detect RBD binding antibodies and neutralizing antibodies in the serum of cynomolgus monkeys after vaccination.

RBD binding antibodies were measured using standard bridged ELISA methods. The specific detection method was as follows: a 96 well ELISA plate was coated with 1 μg/mL of RBD-His at 4° C. for overnight. After washing three times with PBST buffer (containing 0.05% Tween-20 in PBS), the ELISA plate was blocked with 200 L of 1% BSA solution at 37° C. for 1 hour. After washing, the 96 well plate was added with 100 μL serum gradient diluent and incubated at 37° C. for 1 hour. After washing, the 96 well plate was added with 100 μL Protein A-HRP (1:5000 when used), and incubated at 37° C. for one hour and shaken at 650 rpm. After washing, the 96 well plate was incubated with 100 μL of tetramethylbenzidine (TMB) substrate solution at 37° C. and shaken at 650 rpm for 20 minutes. 2M sulfuric acid solution was added to terminate the reaction, and the absorbance value was measured at 450 nm by using the automatic microplate reader SpectraMax.

Neutralizing antibodies were detected using competitive ELISA method. The specific determination method was as follows: the sample and control substance were pre-incubated with HRP-RBD to bind the tested neutralizing antibody to HRP-RBD. Then the mixture was added to the capture plate pre-coated with hACE2 protein. Unbound HRP-RBD and HRP-RBD that bound to non-neutralizing antibodies would be captured on the plate, while the neutralizing antibodies/HRP-RBD complexes in the sample remained in the supernatant and were removed during the washing process. After the washing step, TMB solution was added to turn the color blue. By adding a termination solution, the reaction was quenched and the color turned yellow. This final solution could be read at 450 nanometers in a microplate reader. The absorbance of the sample was inversely proportional to the titer of the neutralizing antibody against SARS-CoV-2.

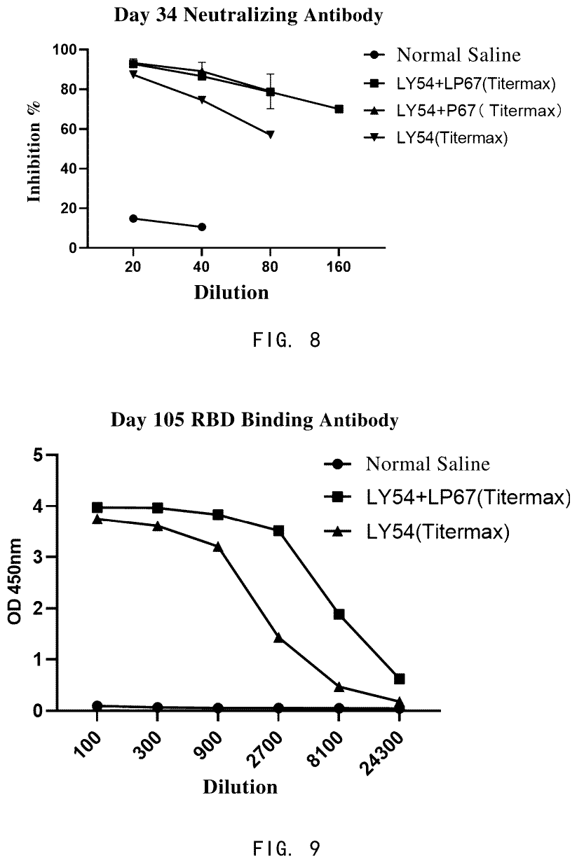

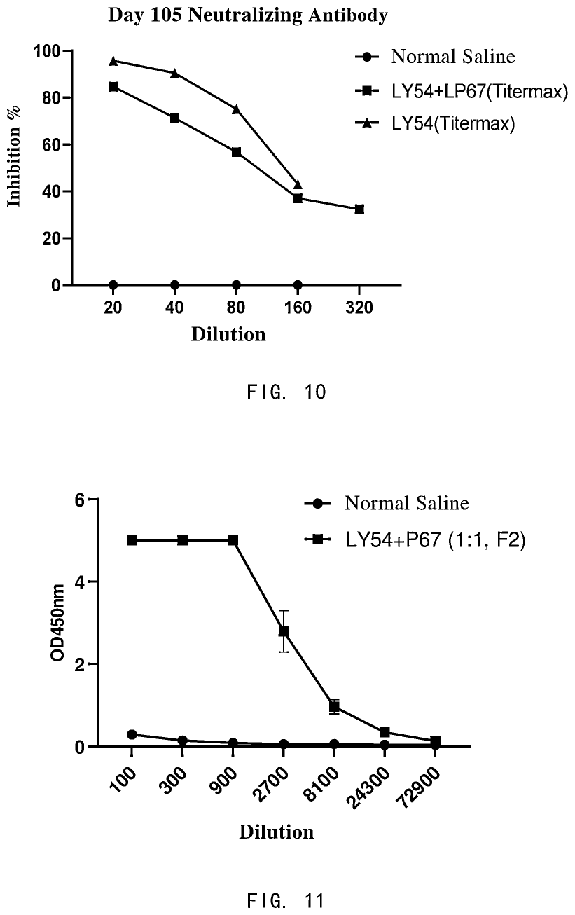

The results showed that the cynomolgus monkeys immunized with polypeptide vaccines produced high levels of RBD specific binding antibodies. On the 34th day after immunization, the titer of RBD specific binding antibodies in the serum of cynomolgus monkeys immunized with polypeptide vaccines reached 1:72900 (FIG. 7). Moreover, polypeptide vaccine immunization induced a high level of neutralizing antibodies that blocked the binding of RBD and ACE2. On the 34th day after immunization, the neutralizing antibody titer in the serum of cynomolgus monkeys immunized with polypeptide vaccines reached 1:160 (FIG. 8). Surprisingly, even after three months of immunization (on the 105th day) to detect the serum antibodies in cynomolgus monkeys, the serum of cynomolgus monkeys still retained RBD specific binding antibodies at a titer of up to 1:24300 (FIG. 9). Surprisingly, three months later, the neutralizing antibody titer in the serum of cynomolgus monkeys did not decrease but instead increased, reaching as high as 1:320 (FIG. 10).

Therefore, the polypeptide vaccine of the present invention induces a high-level, SARS-CoV-2 specific, and persistent humoral immune response with blocking activity in cynomolgus monkeys.

### Example 5 Development of Polypeptide Vaccine Nanoemulsion Formulation

In a fixed amount of polypeptides, 2.0-5.0% squalene, 0.0-4.0% emulsifiers, and 0-20% acceptable raw materials in the injection form were added to prepare nanoemulsions with a droplet size less than 220 nm, meeting the requirements of filtration and sterilization.

Among them, the optimal formula is as follows:

| | | | Methoxypoly- | |
|---|---|---|---|---|
| | | | ethylene | |
| | | | glycol-polylactic | Add aqueous |
| | | | acid block | phase |
| Squalene | Tocopherol | Polysorbate | copolymer % | to 100% |
| % (w/w) | % (w/w) | 80% (w/w) | (w/w) | (w/w) |
| 2.5 | 2.5 | 1.8 | 0.05 | PBS buffer |

Nanoemulsion vaccines with different doses and types of polypeptides as antigens (Table 1) were prepared by using this formula (named F2).

TABLE 1
Different formulas of nanoemulsion vaccines with different
doses and types of polypeptides as antigens

| | Polypeptide | Dose | Particle |
|---|---|---|---|
| Formulation | types | (mg/kg) | size (nm) |
| 1 | LY54 | 1.0 | <220 |
| 2 | LY54 | 2.0 | <220 |
| 3 | P67 | 1.0 | <220 |
| 4 | P67 | 2.0 | <220 |
| 5 | LY54 | 2.0 | <220 |
| | P67 | 1.0 | |
| 6 | MP477 | 1.4 | <220 |
| | MP484 | 1.2 | |
| | MP501 | 0.8 | |
| | P67 | 1.2 | |

According to the particle size in Table 1, the optimized formula can adapt to different types and different doses of polypeptides, and the particle size of nanoemulsion is less than 220 nm, which meets the requirements of filtration and sterilization.

### Example 6 Evaluation of In Vivo Immune Efficacy of Polypeptide Vaccine Nanoemulsion Formulation

1 mg each of LY54 and P67 was used to prepare a nanoemulsion (LY54+P67 (1:1, F2)) according to the formula (F2) in Example 5. Cynomolgus monkeys were divided into groups for intramuscular injection and immunized on days 0, 14, and 28.

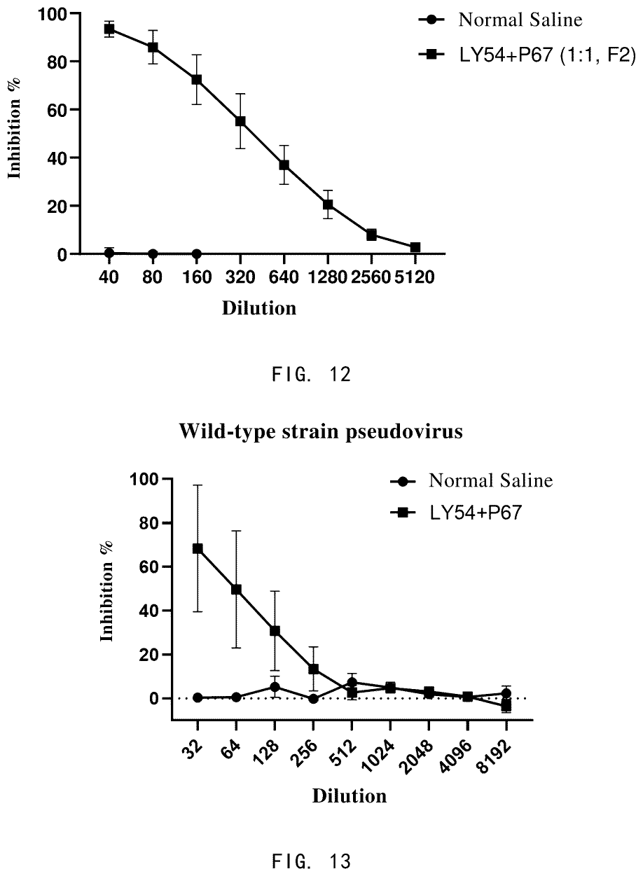

The detection of RBD specific binding antibodies and neutralizing antibodies in the serum of immunized cynomolgus monkeys showed that the polypeptide vaccine nanoemulsion formulation induced a high level of humoral immune response after 42 days of immunization. The titer of RBD binding antibodies in the serum reached 1:24300 (FIG. 11), and the titer of neutralizing antibody reached 1:1280 (FIG. 12). This indicates that the polypeptide vaccine nanoemulsion formulation of the present invention can induce high-level protective humoral immune responses in vivo, and is expected to protect the body from viral infections.

### Example 7 Pseudovirus Neutralization Experiment of Antiserum of Cynomolgus Monkeys Immunized with Polypeptide Vaccine Nanoemulsion

In order to further evaluate whether the high-level humoral immune response induced by polypeptide vaccine nanoemulsion immunization of cynomolgus monkeys can block virus invasion of host cells, a pseudovirus testing system was used to evaluate the pseudovirus blocking and neutralizing activity of cynomolgus monkey antiserum.

In the pseudovirus neutralization test, 100 μL of serum samples with different dilutions were mixed with 50 μL of supernatant containing SARS-CoV-2 wild-type pseudovirus. The mixture was incubated at 37° C. for 1 hour. Then 100 μL of Huh-7/ACE2 cells were added to the mixture of pseudovirus and serum samples, and incubated at 37° C. for another 24 hours. Then, the supernatant was removed and 100 μL of luciferase detection solution was added to each well. After incubation for 2 minutes, the luciferase activity was measured using a microplate photometer.

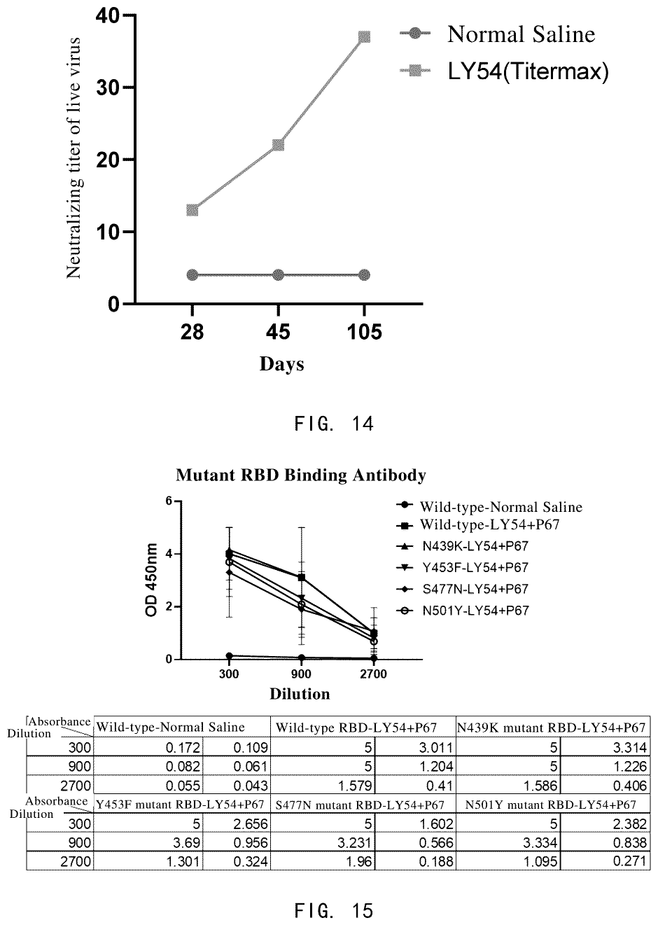

The results showed that the serum of cynomolgus monkeys immunized with the polypeptide vaccine nanoemulsion had a high level of neutralization activity, which could block the invasion of the wild-type pseudovirus into host cells, with a titer of up to 1:256 (FIG. 13).

### Example 8 Live Virus Neutralization Experiment of Antiserum of Cynomolgus Monkeys Immunized with Polypeptide Vaccine Nanoemulsion

In this example, in order to further realistically simulate whether the high-level humoral immune response induced by polypeptide vaccine nanoemulsion immunization of cynomolgus monkeys can block virus invasion of host cells, the live virus cytopathic effect assay was used to evaluate the live virus blocking and neutralizing activity of cynomolgus monkey antiserum.

The wild-type strain of SARS-CoV-2 virus proliferated in VERO E6 cells. Serum samples (antiserum produced by cynomolgus monkeys immunized with polypeptide vaccine formulation LY54 (Titermax)) were thermally inactivated at 56° C. for 30 minutes. Then the twice continuous dilution starting from 1:4 was mixed with an equal volume of virus solution containing 50% tissue culture. The serum-virus mixture was incubated in an environment humidified with 5% CO2 at 37° C. for 1 hour. After incubation, 100 μL mixture of each dilution was added in duplicate to the cell plate containing a semi fused VERO E6 monolayer. The plate was incubated at 37° C. for 4 hour. After 4 days of culture, the cytopathic effect (CPE) of each well was recorded under a microscope. The highest serum dilution that could protect more than 50% of cells from CPE was taken as the neutralization titer.

The results showed that the serum of cynomolgus monkeys immunized with the polypeptide vaccine nanoemulsion had a high level of neutralization activity even 3 month after immunization, which could block the invasion of the live virus into host cells, with a titer of up to 1:37 (FIG. 14).

### Example 9 Polypeptide Vaccine Nanoemulsion Induced Humoral Immune Response in Response to SARS-CoV-2 Mutant Strains

The above results indicate that polypeptide vaccine nanoemulsions can induce high-level, protective, and persistent humoral immune responses against wild-type SARS-CoV-2. In order to evaluate whether the humoral immune response induced by polypeptide vaccines can respond to SARS-CoV-2 mutant strains, the efficacy of polypeptide vaccines was tested by using multiple mutant RBD proteins and the B.1.1.7 mutant pseudovirus system in the present invention.

Firstly, the RBD binding antibody detection method based on Example 4 was used to detect various mutant RBD proteins that replaced wild-type RBD with N439K mutation, Y453F mutation, S477N mutation, and N501Y mutation.

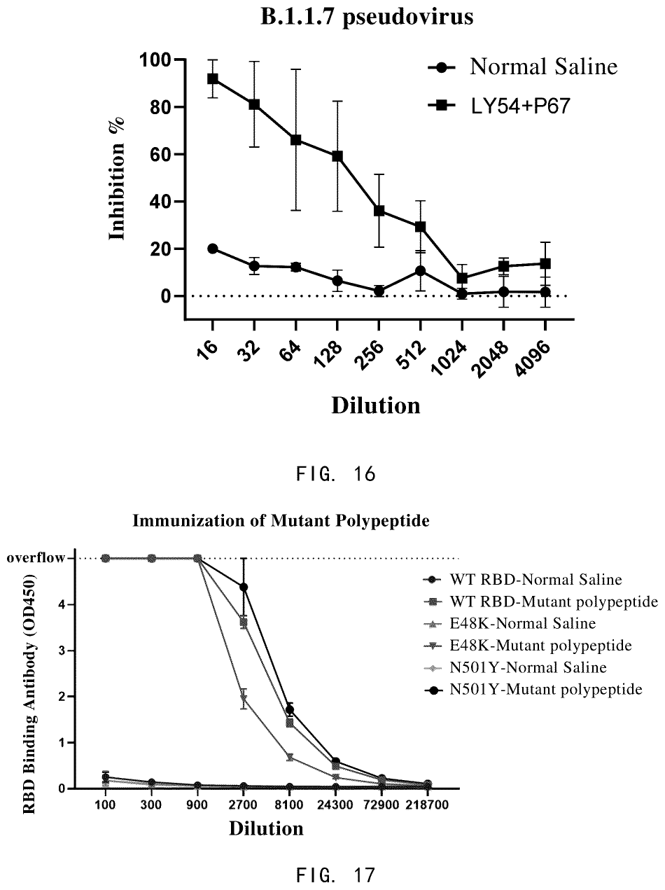

The results showed that after immunization of cynomolgus monkeys with polypeptide vaccine nanoemulsion LY54+P67 (1:1, F2) on day 29, there was no difference in the binding ability of cynomolgus monkey antiserum to wild-type RBD, compared to the binding ability of cynomolgus monkey antiserum to mutant RBD proteins with N439K mutation, Y453F mutation, S477N mutation, or N501Y mutation (FIG. 15). This indicates that the high-level humoral immune response induced by polypeptide vaccine immunization of cynomolgus monkeys is not affected by the above RBD protein mutations, and mutations in the RBD protein will not cause immune escape from the polypeptide vaccine of the present invention.

Furthermore, the blocking activity of the antiserum of cynomolgus monkeys immunized with the polypeptide vaccine nanoemulsion against the currently widely prevalent mutant strain B.1.1.7 was tested in the present invention. The pseudovirus system of B.1.1.7 was adopted, and the basic method was as described in Example 5. Testing showed that the antiserum of cynomolgus monkeys had a high level of blocking activity against the mutant strain B.1.1.7, with a titer of up to 1:512 (FIG. 16). Comparing the neutralizing effect of cynomolgus monkey antiserum on wild-type pseudoviruses, it indicates that mutant strain B.1.1.7 does not cause immune escape to the polypeptide vaccine of the present invention, and the polypeptide vaccine of the present invention can protect the body from infection by the mutant strain.

In order to further improve the protective effect of the polypeptide vaccine nanoemulsion of the present invention on novel coronavirus mutants, the polypeptides MP477, MP484 and MP501 screened and determined against SARS-CoV-2 mutant strains in Example 1 were mixed with P67 to form a combined epitope polypeptide, which was then prepared into a nanoemulsion and used to immune cynomolgus monkeys. Cynomolgus monkeys were immunized by intramuscular injection on day 0 and day 14, and blood samples were collected on day 21 (7 days after the second immunization) to detect mutant RBD specific binding antibodies in the serum.

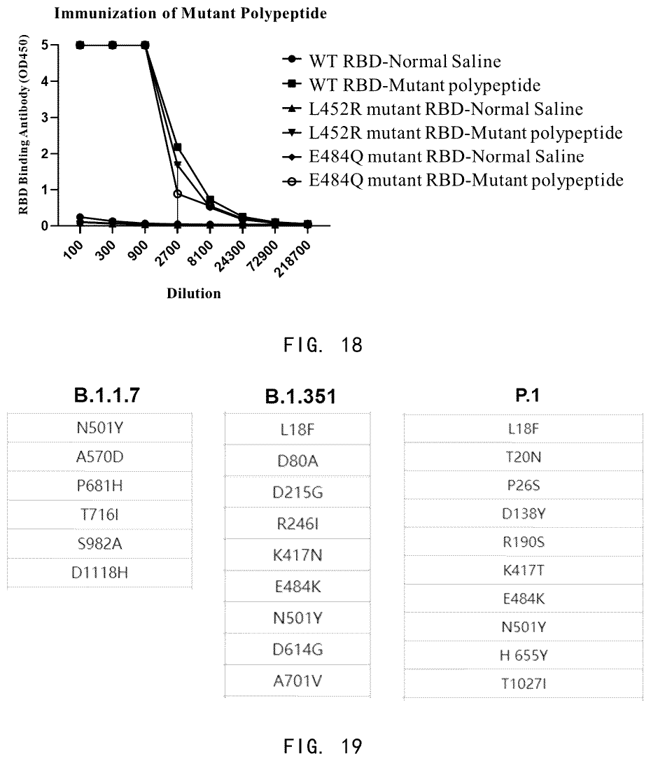

Surprisingly, the high-level RBD binding antibodies in immunized cynomolgus monkeys, induced by the vaccine polypeptide against the mutant strains, also had a high level of binding activity to the E484K mutant RBD with the immune escape effect of novel coronavirus vaccines and neutralizing antibody drugs (FIG. 17), suggesting that the polypeptide vaccine nanoemulsion could protect the body from the infection of the mutant strain B.1.351 and mutant strain P.1 with E484K mutation.

In addition, the high-level RBD binding antibodies in immunized cynomolgus monkeys, induced by the vaccine polypeptide against the mutant strains, also had a high level of binding activity to the RBD mutated at the key mutation sites L452R and E484Q of the B.1.617 mutant strain with the immune escape effect of novel coronavirus vaccines and neutralizing antibody drugs (FIG. 18), suggesting that the polypeptide vaccine nanoemulsion could protect the body from the infection of the B.1.617 mutant strain with L452R and E484Q mutations.

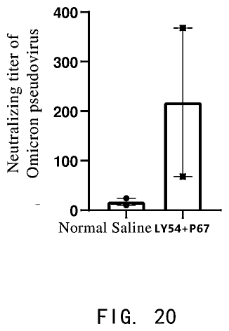

Meanwhile, using the Omicron (B.1.1.529) pseudovirus model, the antiserum of this vaccine (7 days after the third immunization) was also identified to have the ability to neutralize the Omicron mutant strain, with a neutralizing titer of 1:218 (FIG. 20), indicating that the polypeptide vaccine nanoemulsion could protect the body from infection by the Omicron mutant strain.

### DISCUSSION

Polypeptide vaccines are a type of vaccines that can be quickly designed and mass-produced against pathogen proteins, and they are safe in nature, specific in action, and low in cost. Therefore, polypeptide vaccines are suitable for responding to sudden epidemics and pathogen mutations.

At present, all the novel coronavirus vaccines, which have passed clinical trials, are designed based on the SARS-CoV-2 wild-type strains. Previous studies have shown that the currently prevalent strains of B.1.1.7 mutant strain, B.1.351 mutant strain, P.1 mutant strain, B.1.617 mutant strain and B.1.1.529 mutant strain have the nature of immune escape, which may make the existing novel coronavirus vaccines and neutralizing antibody drugs ineffective and reduce their protective effects. Therefore, there is an urgent need for vaccine products that can deal with novel coronavirus mutant strains. The technical route of the polypeptide vaccine combination of the present invention is different from the technical routes of the existing novel coronavirus vaccines, and is matched with the optimized nanoemulsion adjuvant formulation. The immune effect of the polypeptide nanoemulsion vaccine of the present invention is still efficient against novel coronavirus mutant strains, and the blocking effect has been verified on the pseudovirus and live virus systems of wild-type strains and mutant strains, thus the polypeptide nanoemulsion vaccine of the present invention has the development prospect of responding to the prevalence of mutant strains.

All references mentioned in the present application are incorporated by reference herein, as though individually incorporated by reference. In addition, it should be understood that after reading the above teaching content of the present invention, various changes or modifications may be made by those skilled in the art, and these equivalents also fall within the scope as defined by the appended claims of the present application.

Sequences of Vaccine polypeptides

>SEQ ID NO: 1 LY54
LFRKSNLKPFERDISTEIYQAGSTPCNGVEGENCYFPLQSYGFQPTNG
VGYQPY
>SEQ ID NO: 2 P67
YAWNRKRISNCVADYSVLYNSASFSTFK
>SEQ ID NO: 3 PSS
YAWNRKRISNC(C)VADYSVLYNSASFSTFK
>SEQ ID NO: 4 LP67
ISNCVADYSVLYNSASFSTFKCYGVSPTKLNDLCFTNV
>SEQ ID NO: 5 LPSS
ISNC(C)VADYSVLYNSASFSTFKC(C)YGVSPTKLNDLC(C)FTNV
>SEQ ID NO: 6 MP1
VGGNYNYLYRLFRKSNLKPFERDISTEIYQAGSTP
>SEQ ID NO: 7 MP2
DISTEIYQAGSTPCNGVEGENCYFPLQSYG
>SEQ ID NO: 8 MP3
YFPLQSYGFQPTNGVGYQPY
>SEQ ID NO: 9 MP477
VGGNYNYLYRLFRKSNLKPFERDISTEIYQAGNTP
>SEQ ID NO: 10 MP484
DISTEIYQAGSTPCNGVKGFNCYFPLQSYG
>SEQ ID NO: 11 MP501
YFPLQSYGFQPTYGVGYQPY
>SEQ ID NO: 12 LY54mut
LFRKSNLKPFERDISTEIYQAGSTPCNGVKGFNCYFPLQSYGFQPTYG
VGYQPY
>SEQ ID NO: 13 NTD1
QCVNLTTRTQLPPAYTNSFTRG
>SEQ ID NO: 14 NTD2
CNDPFLGVYYHKNNKSWMESEFRVYS
>SEQ ID NO: 15 NTD3
LHRSYLTPGDSSSGWTAGAAAY
(Note: SEQ ID Nos: 13-15 are derived from the NTD region of S protein)

1. A vaccine polypeptide against coronavirus SARS-CoV-2, wherein the vaccine polypeptide has an amino acid sequence derived from the S protein of SARS-CoV-2 wild-type and mutant strains;
and the vaccine polypeptide is selected from the group consisting of:
(a) a polypeptide having the amino acid sequence shown in any one of SEQ ID NOs: 1-15;
(b) a derivative polypeptide formed by one or more amino acids addition, one or more amino acids substitution, or 1-3 amino acids deletion to the amino acid sequence of the polypeptide in (a), and the derivative polypeptide has essentially identical function to the original polypeptide before derivatization.

2. The vaccine polypeptide of claim 1, wherein the vaccine polypeptide can induce primates to produce wild-type and mutant RBD specific binding antibodies and neutralizing antibodies that block the binding of wild-type and mutant RBD to ACE2.

3. The vaccine polypeptide of claim 1, wherein the SARS-CoV-2 mutant strains are selected from the group consisting of: D614G mutant strain, B.1.1.7 mutant strain, B.1.351 mutant strain, P.1 mutant strain, B.1.617 mutant strain, B.1.1.529 mutant strain, and a combination thereof.

4. The vaccine polypeptide of claim 1, wherein the structure of the vaccine polypeptide is as shown in Formula I:
X1-X-X2 (I),
wherein,
(a) X is a core fragment, wherein the sequence of the core fragment is selected from one or more of SEQ ID NOs: 1-15 (see Table A);
(b) X1 and X2 each independently represent none, 1, 2, or 3 amino acids, and the total number of amino acids of X1 and X2 is ≤4, preferably is 3, 2, or 1, and more preferably is 0 or 1;
(c) “-” represents a peptide bond, peptide linker, or other linker (that is, X1 and X, and/or, X and X2 are connected by peptide bonds, peptide linkers (such as a flexible linker consisting of 1-15 amino acids) or other linkers).

5. The vaccine polypeptide of claim 1, wherein the vaccine polypeptide is selected from Table A:
TABLE A
Vaccine polypeptides

| Peptide | | SEQ |
|---|---|---|
| ID | Sequence | ID No: |
| LY54 | LFRKSNLKPFERDISTEIYQAGSTPCNGVEGENCY | 1 |
| | FPLQSYGFQPTNGVGYQPY | |
| P67 | GYAWNRKRISNCVADYSVLYNSASFSTFK | 2 |
| PSS | GYAWNRKRISNC(C)VADYSVLYNSASFSTFK | 3 |
| LP67 | ISNCVADYSVLYNSASFSTFKCYGVSPTKLNDLCF | 4 |
| | TNV | |
| LPSS | ISNC(C)VADYSVLYNSASFSTFKC(C)YGVSPTK | 5 |
| | LNDLC(C)FTNV | |
| MP1 | VGGNYNYLYRLFRKSNLKPFERDISTEIYQAGSTP | 6 |
| MP2 | DISTEIYQAGSTPCNGVEGENCYFPLQSYG | 7 |
| MP3 | YFPLQSYGFQPTNGVGYQPY | 8 |
| MP477 | VGGNYNYLYRLFRKSNLKPFERDISTEIYQAGNTP | 9 |
| MP484 | DISTEIYQAGSTPCNGVKGFNCYFPLQSYG | 10 |
| MP501 | YFPLQSYGFQPTYGVGYQPY | 11 |
| LY54mut | LFRKSNLKPFERDISTEIYQAGSTPCNGVKGFNCY | 12 |
| | FPLQSYGFQPTYGVGYQPY | |
| NTD1 | QCVNLTTRTQLPPAYTNSFTRG | 13 |
| NTD2 | CNDPFLGVYYHKNNKSWMESEFRVYS | 14 |
| NTD3 | LHRSYLTPGDSSSGWTAGAAAY | 15 |

wherein, the “C(C)” in SEQ ID Nos: 3 and 5 indicates that the cysteine on the peptide chain forms a disulfide bond with another cysteine on the non-peptide chain.

6. The vaccine polypeptide of claim 4, wherein X1 or X2 each independently represents none, K, C, G, L, or A.

7. The vaccine polypeptide of claim 1, wherein the vaccine polypeptide has at least one T cell epitope and/or at least one B cell epitope of the SARS-CoV-2 S protein.

8. An isolated peptide set, wherein the peptide set comprises at least two vaccine polypeptides against coronavirus SARS-CoV-2 of claim 1.

9. The isolated peptide set of claim 8, wherein the peptide set comprises the vaccine polypeptide selected from the group consisting of:
(Z1) 1 or 2 vaccine polypeptides selected from SEQ ID Nos: 1 and 12, and 1, 2, 3, or 4 vaccine polypeptides selected from SEQ ID Nos: 2-5;
(Z2) 1 or 2 vaccine polypeptides selected from SEQ ID Nos: 6 and 9, 1 or 2 vaccine polypeptides selected from SEQ ID Nos: 7 and 10, and 1 or 2 vaccine polypeptides selected from SEQ ID Nos: 8 and 11;
(Z3) one or more vaccine polypeptides selected from SEQ ID Nos: 1-12, and 1, 2 or 3 vaccine polypeptides selected from SEQ ID Nos: 13-15.

10. The isolated peptide set of claim 8, wherein the peptide set comprises the vaccine polypeptide selected from the group consisting of:
(s1) the vaccine polypeptide shown in SEQ ID No: 1, and the vaccine polypeptide shown in SEQ ID No: 2;
(s2) 1 or 2 vaccine polypeptides selected from SEQ ID Nos: 1 and 12, and 1, 2, 3 or 4 vaccine polypeptides selected from SEQ ID Nos: 2-5, and the peptide set at least comprises one or more vaccine polypeptides selected from SEQ ID Nos: 3, 4, 5, and 12;
(s3) the vaccine polypeptides shown in SEQ ID Nos: 6, 7 and 8;
(s4) the vaccine polypeptides shown in SEQ ID Nos: 9, 10 and 11;
(s5) the vaccine polypeptides shown in SEQ ID Nos: 6, 10 and 11;
(s6) the vaccine polypeptides shown in SEQ ID Nos: 9, 7 and 11;
(s7) the vaccine polypeptides shown in SEQ ID Nos: 9, 10 and 8;
(s8) the vaccine polypeptides shown in SEQ ID Nos: 2, 9, 10 and 11;
(s9) the vaccine polypeptides shown in SEQ ID Nos: 2, 6, 7 and 8.

11. A pharmaceutical composition comprising:
the vaccine polypeptide against coronavirus SARS-CoV-2 of claim 1 or a peptide set comprising at least two of the vaccine polypeptides against coronavirus SARS-CoV-2, and a pharmaceutically acceptable carrier.

12. A vaccine polypeptide nanoemulsion formulation against coronavirus SARS-CoV-2, which comprises:
(a) the vaccine polypeptide against coronavirus SARS-CoV-2 of claim 1;
(b) an adjuvant, preferably the adjuvant is an oil-in-water nanoemulsion based on squalene; and
(c) a pharmaceutically acceptable carrier, excipient or diluent.

13. (canceled)

14. A cell preparation, which comprises (a) immune cells which are immune activated by the vaccine polypeptide against coronavirus SARS-CoV-2 of claim 1 or a peptide set comprising at least two of the vaccine polypeptides against coronavirus SARS-CoV-2; and (b) a pharmaceutically acceptable carrier.

15. A method for generating an immune response to the coronavirus SARS-CoV-2 wild-type and mutant strains, which comprises the step of: administering the vaccine polypeptide against coronavirus SARS-CoV-2 of claim 1, or a peptide set comprising at least two of the vaccine polypeptides against coronavirus SARS-CoV-2, to a subject in need thereof.

16. The vaccine polypeptide nanoemulsion formulation of claim 12, wherein the formulation comprises a nanoemulsion based on squalene and an emulsifier, and the squalene is derived from shark liver, and the emulsifier is phospholipid, or polysorbate 80, or a combination of polysorbate 80 and phospholipids, as well as one or more of sucrose esters, citric acid fatty acid glycerides, fatty acid glycerides, fatty acid sorbitans, cyclodextrins, polyoxyethylene fatty acid esters.
